(19) United States
(12) Patent Application Publication
(10) Pub. No.: US 2011/0137116 A1
(43) Pub. Date: Jun. 9, 2011

(54) Endoscopic Surgical Instrument With Color-Coded Working Parts

(76) Inventors: Helmut Wehrstein, Tuttlingen (DE); Heiko Baehr, Dauchingen (DE); Uwe Bacher, Tuttlingen (DE)

(21) Appl. No.: 13/027,967

(22) Filed: Feb. 15, 2011

Related U.S. Application Data
(63) Continuation of application No. 11/029,796, filed on Jan. 5, 2005, now abandoned.

(30) Foreign Application Priority Data
Jul. 5, 2004 (EM) 000199716

(51) Int. Cl. A61B 1/018

(52) U.S. Cl. 600/106

(57) ABSTRACT

An endoscopic surgical instrument includes an insertion portion adapted to be inserted into a body, the insertion portion having a proximal end extending outside the body during use and a distal end located within the body during use, a working part attached to the distal end of the insertion portion, the working part being disposed within the body during use, and a handle portion attached to the proximal end of the insertion portion, the handle portion being disposed outside of the body during use, the handle portion being actuatable to actuate the working part via a force transmission member. The working part is a member of one of a plurality of subsets of working parts, members of each subset of working parts being visually distinguishable from members of each other subset of working parts by color.

### CROSS-REFERENCE TO RELATED APPLICATIONS

The present application is a continuation of pending U.S. patent application Ser. No. 11/029,796 filed on Jan. 5, 2005, which claims priority to European Design Patent Application No. 00199716 filed Jul. 5, 2004. The content of all prior applications is incorporated herein by reference.

### FIELD OF THE INVENTION

The present invention relates generally to an improved surgical instrument which is used in a surgical procedure, particularly an endoscopic surgical procedure, and more specifically to an improved surgical instrument of the type that includes an actuating working part at a distal end thereof and a handle at a proximal end thereof for causing actuation of the working part.

### BACKGROUND OF THE INVENTION

It has become common to use endoscopes in order to perform various types of treatments on treatment sites while observing an object image with the endoscope (which has an insertion portion formed as an elongated shaft) inserted into body cavities or the like. More specifically, surgical procedures are now common in which therapeutic measures or treatments are performed under endoscopic observation without opening the body of a patient in order to reduce the degree of invasion of the patient's body. In such surgical procedures, an endoscope used for observation is guided into a body cavity via a trocar, and a surgical instrument is guided to the treatment side inside the body cavity through this or another trocar. Then, a therapeutic procedure or treatment is performed while observing the surgical instrument and treatment site by means of the endoscope.

Many surgical instruments for use in these types of endoscopic surgical procedures have been developed. Such surgical instruments are generally long and slender, and are designed so that a surgical treatment part (hereafter also referred to as a “working part”) disposed on the distal end of an insertion portion is actuated by operating an actuating handle disposed on the proximal end of the insertion portion. Actuating motion of the handle is transferred by a force transmitting mechanism through the insertion portion to the working part.

While such endoscopic surgical instruments have long been known, and while many designs for such endoscopic surgical instruments have been created, deficiencies still exist.

Typically, during an endoscopic procedure the endoscopic surgical instruments for the procedure are provided on a table or surgical tray from which the surgeon and/or the surgeon's assistants must choose during the procedure. Often, the surgeon does not know precisely which instruments will be needed until the surgery has commenced, and as such, all possibly needed instruments may be provided on the table or surgical tray, thereby possibly rendering the number of instruments provided very large. This fact may make it difficult for the surgeon and/or the surgeon's assistants to identify a particular desired endoscopic surgical instrument or type of instrument from among the large number of instruments provided on the table or tray. Such can be particularly disadvantageous in instances where a particular desired endoscopic surgical instrument is needed quickly, such as where the health of the patient may be deleteriously affected if the proper instrument is not readily identified.

In addition, particularly in cases where the surgery itself is monitored via a video endoscope system having a video monitor display or the like, a surgeon may have trouble readily identifying the type of endoscopic surgical instrument which is currently inserted into the patient's body (particularly if the instrument had been originally inserted into the body by another surgeon or by a surgeon's assistant) by the shape or design of the working part alone. This is particularly true when the working part of the endoscopic surgical instrument is fully or partially covered in blood and/or other bodily fluids or tissues.

Moreover, after the surgical procedure is completed, the endoscopic surgical instruments are typically disassembled, cleaned and disinfected, and then reassembled. During reassembly, however, it is often difficult to identify, from the often large number of instrument parts, particular parts of the various instruments which belong together. Such may be particularly problematic when a large number of instruments were used during the surgical procedure and/or when a large number of instruments from two or more surgical procedures are being cleaned and disinfected together.

What is desired, therefore, is an endoscopic surgical instrument which is readily distinguishable as a particular desired endoscopic surgical instrument or type of endoscopic surgical instrument from among other endoscopic surgical instruments provided together on a table or tray or the like, which is readily identifiable as a particular endoscopic surgical instrument or type of endoscopic surgical instrument while it is inserted into a patient's body, and which includes parts which are readily distinguishable from the parts of other endoscopic surgical instruments during reassembly, after the endoscopic surgical instrument, along with other endoscopic surgical instruments, have been disassembled and cleaned and disinfected.

### SUMMARY OF THE INVENTION

Accordingly, it is an object of the present invention to provide an endoscopic surgical instrument which is readily distinguishable as a particular desired endoscopic surgical instrument or type of endoscopic surgical instrument from among other endoscopic surgical instruments provided together on a table or tray or the like.

Another object of the present invention is to provide an endoscopic surgical instrument having the above characteristics and which is readily identifiable as a particular endoscopic surgical instrument or type of endoscopic surgical instrument while it is inserted into a patient's body.

A further object of the present invention is to provide an endoscopic surgical instrument having the above characteristics and which includes parts which are readily distinguishable from the parts of other endoscopic surgical instruments during reassembly, after the endoscopic surgical instrument, along with other endoscopic surgical instruments, have been disassembled and cleaned and disinfected.

These and other objects are achieved according to one embodiment of the present invention by provision of an endoscopic surgical instrument including an insertion portion adapted to be inserted into a body, the insertion portion having a proximal end extending outside the body during use and a distal end located within the body during use, a working part attached to the distal end of the insertion portion, the working part being disposed within the body during use, and a handle portion attached to the proximal end of the insertion portion, the handle portion being disposed outside of the body during use, the handle portion being actuatable to actuate the working part via a force transmission member. The working part is a member of one of a plurality of subsets of working parts, members of each subset of working parts being visually distinguishable from members of each other subset of working parts by color.

In some embodiments, the working part is formed from a material, and the working part is visually distinguishable from members of each other subset of working parts by a color of the material. In certain of these embodiments, the material comprises at least one of a metal, a plastic and a ceramic. In some embodiments, the working part is visually distinguishable from members of each other subset of working parts by a color which the working part is painted. In some embodiments, the working part in its entirety is visually distinguishable from members of each other subset of working parts by color. In some embodiments, less than an entirety of the working part is visually distinguishable from members of each other subset of working parts by color. In certain of these embodiments, the working part comprises a pair of cooperating jaws, and the pair of cooperating jaws are visually distinguishable from jaws of members of each other subset of working parts by color.

In some embodiments, each subset of the plurality of subsets of working parts comprises at least one working part intended for a particular use. In certain of these embodiments, the working part comprises a member one of the following subsets: graspers, scissors, gripper tools, biopsy tools, dissectors, and needle holders. In some embodiments, each subset of the plurality of subsets of working parts comprises at least one working part intended for use during a particular type of surgical procedure. In some embodiments, each subset of the plurality of subsets of working parts comprises at least one working part intended for use with a particular type of patient.

In accordance with another embodiment of the present invention, a surgical instrument includes a working part and a handle portion attached via an elongated shaft to the working part, the handle portion being manipulatable to actuate the working part via a force transmission member. The surgical instrument comprises a member of one of a plurality of subsets of surgical instruments, and members of each subset of surgical instruments are visually distinguishable from members of each other subset of surgical instruments by color.

In some embodiments, the surgical instrument is formed from a material, and the surgical instrument is visually distinguishable from members of each other subset of surgical instruments by a color of the material. In certain of these embodiments, the material comprises at least one of a metal, a plastic and a ceramic. In some embodiments, the surgical instrument is visually distinguishable from members of each other subset of surgical instruments by a color which the surgical instrument is painted. In some embodiments, the surgical instrument in its entirety is visually distinguishable from members of each other subset of surgical instruments by color. In some embodiments, less than an entirety of the surgical instrument is visually distinguishable from members of each other subset of surgical instruments by color. In certain of these embodiments, the working part is visually distinguishable from working parts of members of each other subset of surgical instruments by color. In certain of these embodiments, the working part comprises a pair of cooperating jaws, and the pair of cooperating jaws are visually distinguishable from jaws of members of each other subset of surgical instruments by color.

In some embodiments, each subset of the plurality of subsets of surgical instruments comprises at least one surgical instrument intended for a particular use. In certain of these embodiments, the surgical instrument comprises a member one of the following subsets: graspers, scissors, gripper tools, biopsy tools, dissectors, and needle holders. In some embodiments, each subset of the plurality of subsets of surgical instruments comprises at least one surgical instrument intended for use during a particular type of surgical procedure. In some embodiments, each subset of the plurality of subsets of surgical instruments comprises at least one surgical instrument intended for use with a particular type of patient.

In accordance with another embodiment of the present invention, a collection of surgical instruments adapted to be used during a surgical procedure includes a first subset of surgical instruments and a second subset of surgical instruments, each of which comprises at least one member which comprises a working part and a handle portion attached via an elongated shaft to the working part, the handle portion being manipulatable to actuate the working part via a force transmission member. The at least one member of the first subset of surgical instruments is visually distinguishable from the at least one member of the second subset of surgical instruments by color.

The invention and its particular features and advantages will become more apparent from the following detailed description considered with reference to the accompanying drawings.

### BRIEF DESCRIPTION OF THE DRAWINGS

FIG. 1 is an isometric side view of an endoscopic surgical instrument in accordance with an embodiment of the present invention;

FIG. 2 is an enlarged, isometric side view of a handle portion of the endoscopic surgical instrument of FIG. 1;

FIG. 3 is an enlarged, partially broken-away, isometric side view of a handle portion of the endoscopic surgical instrument of FIG. 1;

FIG. 4 is an enlarged, isometric side view of a working part portion which may form a part of the endoscopic surgical instrument of FIG. 1;

FIG. 5A is an enlarged, isometric side view of another working part portion which may form a part of the endoscopic surgical instrument of FIG. 1;

FIG. 5B is an enlarged, partially broken-away, side elevational view of the working part portion shown in FIG. 5A;

FIG. 6A is an enlarged, isometric side view of another working part portion which may form a part of the endoscopic surgical instrument of FIG. 1;

FIG. 6B is an enlarged, partially broken-away, side elevational view of the working part portion shown in FIG. 6A; and

FIG. 7 is an enlarged, partially broken-away, side elevational view of a further working part portion which may form a part of the endoscopic surgical instrument of FIG. 1;

FIGS. 8-10 are enlarged, side elevational views of still further working part portions which may form a part of the endoscopic surgical instrument of FIG. 1; and

FIG. 11 is an enlarged, side elevational view, partially in cross-section, of a further working part portion which may form a part of the endoscopic surgical instrument of FIG. 1.

### DETAILED DESCRIPTION OF THE INVENTION

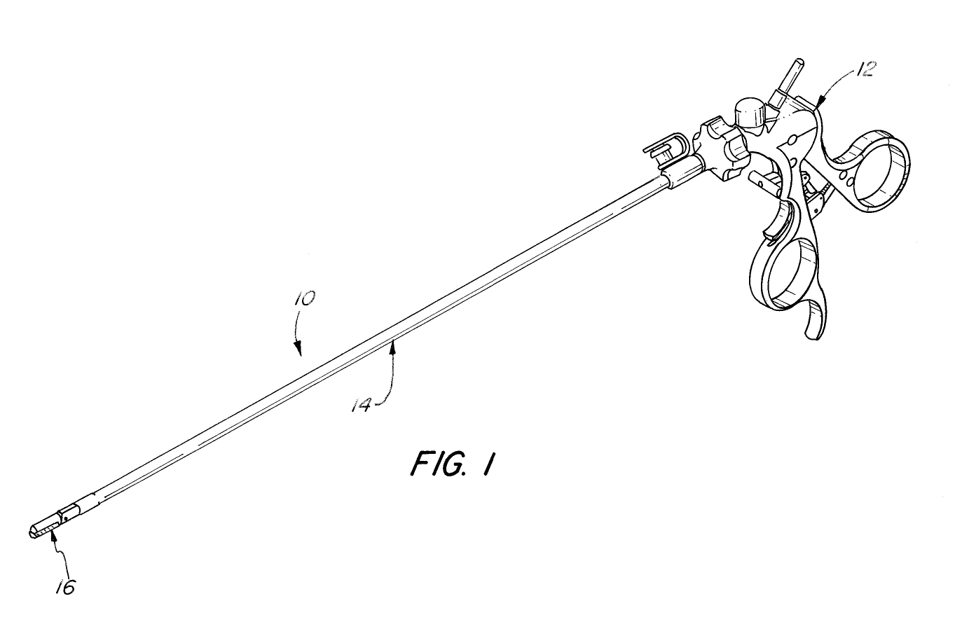

Referring first to FIG. 1, an endoscopic surgical instrument 10 in accordance with an embodiment of the present invention is shown. The instrument 10 can generally be considered as comprising three parts: a handle portion 12, an elongated insertion portion 14 and a working part 16. During use, the working part 16 and at least a portion of the insertion portion 14 are introduced into a body cavity, typically through a trocar, while the handle portion 12 and possibly a portion of the insertion portion 14 remain outside the body. The handle portion 12, which includes an actuator, is manipulated to actuate the working part 16 such that some type of treatment is effected within the body cavity, the particular type of treatment being dependent upon the type of working part 16 employed. The manipulations of working part 16 within the body cavity are viewed using one or more endoscopes introduced through the same trocar as the instrument 10 or through one or more additional trocars.

Instrument 10 may be used in any of a number of different endoscopic surgeries, and may be used with any of numerous types of working parts 16 having various dimensions and configurations, as more fully described below. During a surgery, it is typical that several endoscopic surgical instruments 10 having different working parts 16 may be employed. It is also typical that additional endoscopic surgical instruments 10 which ultimately are not used during the surgical procedure may be provided in the surgical area, as it is often not possible to determine before the surgical procedure commences precisely which endoscopic surgical instruments 10 will be needed. Typically, this group of endoscopic surgical instruments 10 are provided together on a table or surgical tray, with the surgeon and/or the surgeon's assistants selecting instruments 10 from the table or tray as they are needed.

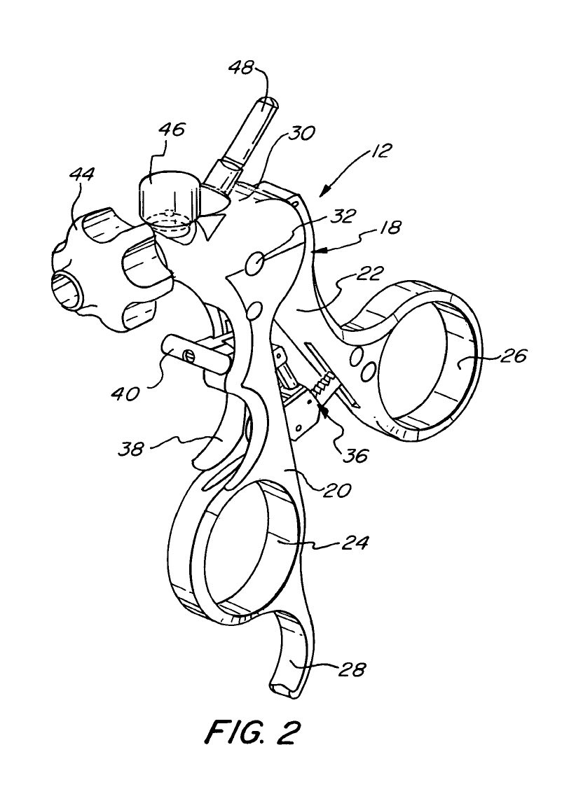

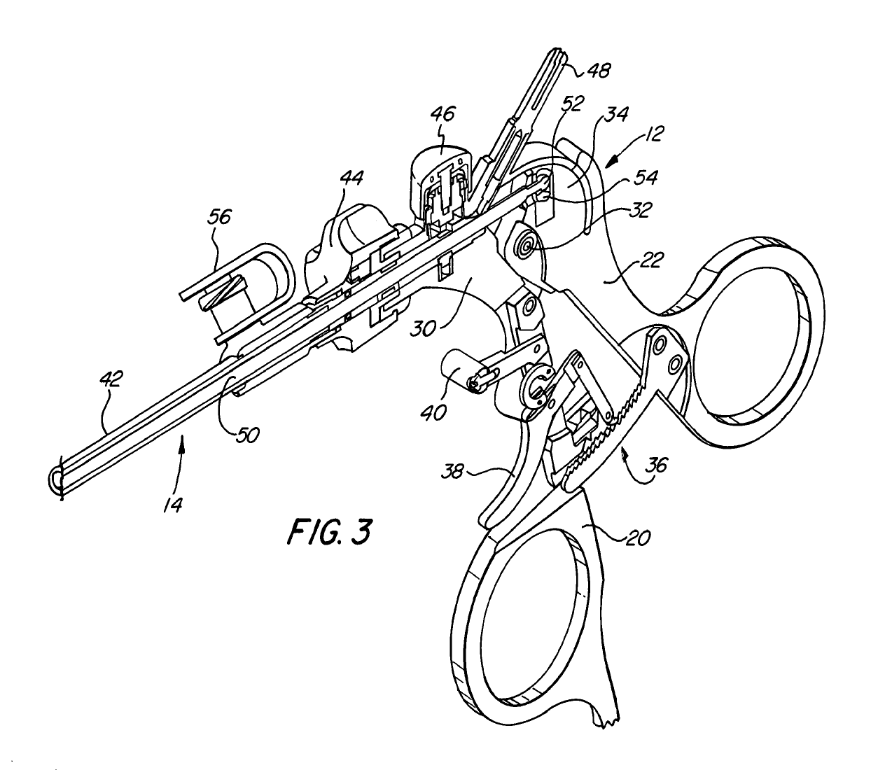

Referring now to FIGS. 2 and 3, handle portion 12 and a portion of insertion portion 14 (FIG. 3) are shown in greater detail. The handle portion 12 includes a scissors component 18 which is gripped by the surgeon and has front and rear scissors handles 20, 22, respectively. Scissors handles 20, 22 include finger loops 24, 26, and front scissors handle 20 includes a finger rest 28. Front scissors handle 20 includes a housing 30 to which scissors handle 22 is pivotably attached by a pivot pin 32. Thus, front scissors handle 20 is generally considered stationary, while rear scissors handle 22 is generally considered pivotable with respect thereto. When rear scissors handle 22 is pivoted relative to front scissors handle 20 about pivot pin 32, an upper end 34 of rear scissors handle 22 reciprocates relative to the housing 30.

Handle portion 12 may include a ratchet mechanism 36 for locking rear scissors handle 22 in an actuated position with respect to front scissors handle 20. A trigger 38 or the like may be provided for disengaging the ratchet mechanism 36 when it is desired to release the rear scissors handle 22. A switch 40, button or the like may be provided to holding the disengaging trigger 38 in the release position such that the ratchet mechanism 36 is not operational.

As shown in FIG. 3, the housing 30 of front scissor handle 20 includes an opening for receiving an elongated hollow shaft 42 of insertion portion 14. Handle portion 12 may include a rotation mechanism 44 which allows for elongated shaft 42 to be rotated with respect to housing 30, and therefore all of handle portion 12. Handle portion 12 may also include a button 46 or the like which causes disengagement of the insertion portion 14 from the handle portion 12, and an electrode pin 48 for receiving a high-frequency current when instrument 10 is capable of use in high-frequency surgical applications.

As mentioned above, insertion portion 14 includes elongated hollow shaft 42. Passing longitudinally through shaft 42 is a force transmitting member 50 which transmits actuating movement of handle portion 12 to the working part 16. More specifically, force transmitting member 50 includes at a proximal end thereof, a ball element 52 which engages a socket element 54 formed in upper end 34 of rear scissors handle 22. Through this arrangement, pivoting movement of rear scissors handle 22 with respect to front scissors handle 20 is translated into reciprocal movement of force transmission member 50 longitudinally with respect to, and within, elongated hollow shaft 42.

Insertion portion 14 may include an irrigation channel therein, which in some embodiments may be defined between an inner surface of shaft 42 and an outer surface of force transmission member 50. An irrigation connection 56 may be provided for attaching an irrigation source in communication with the irrigation channel.

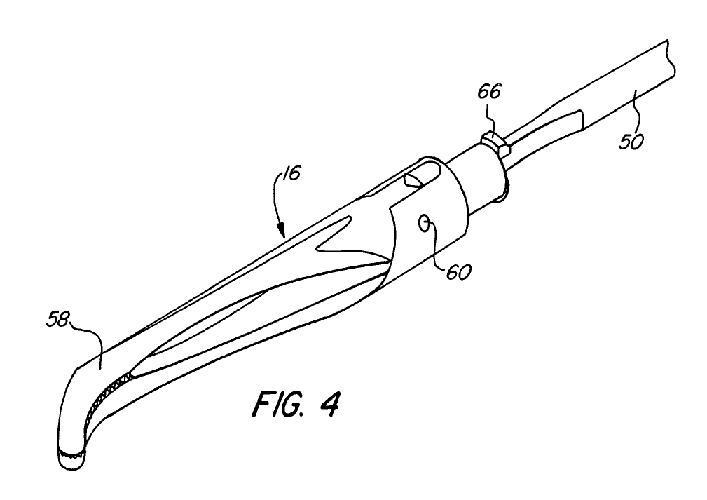

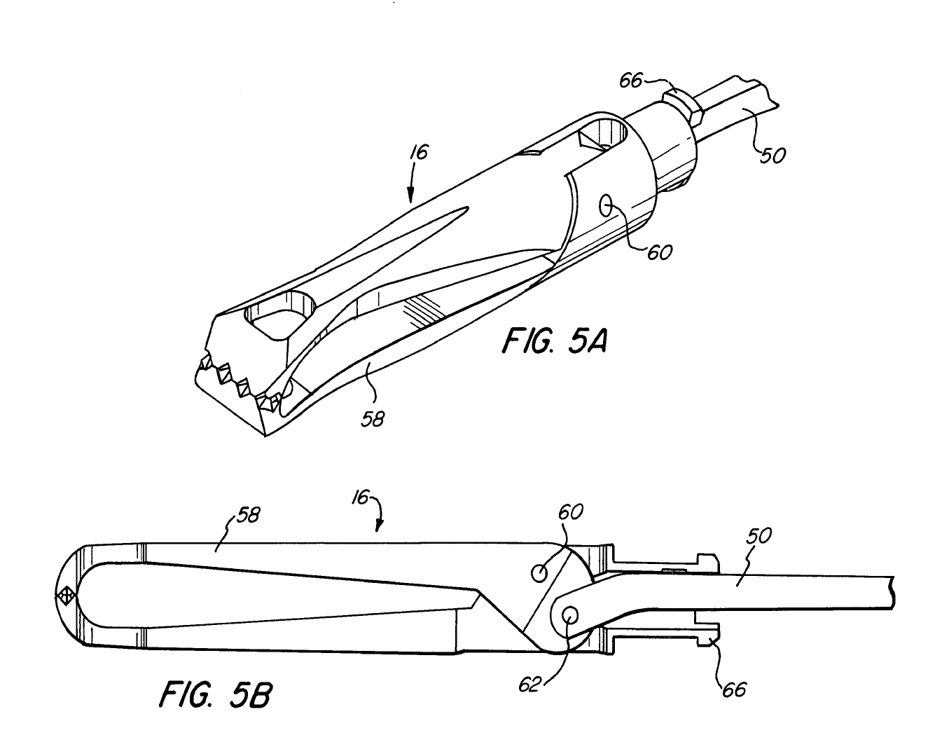

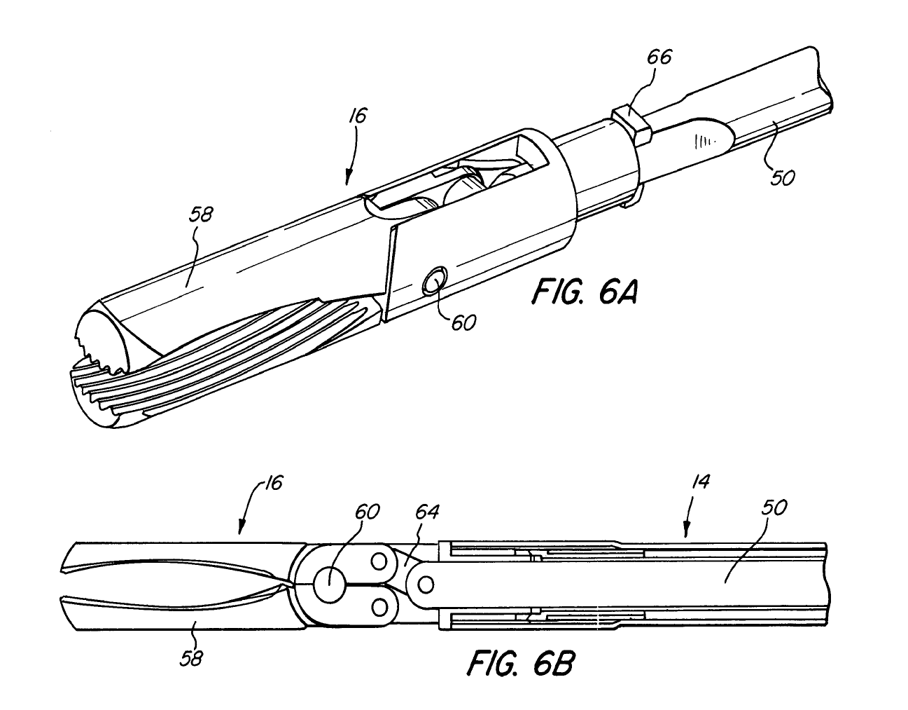

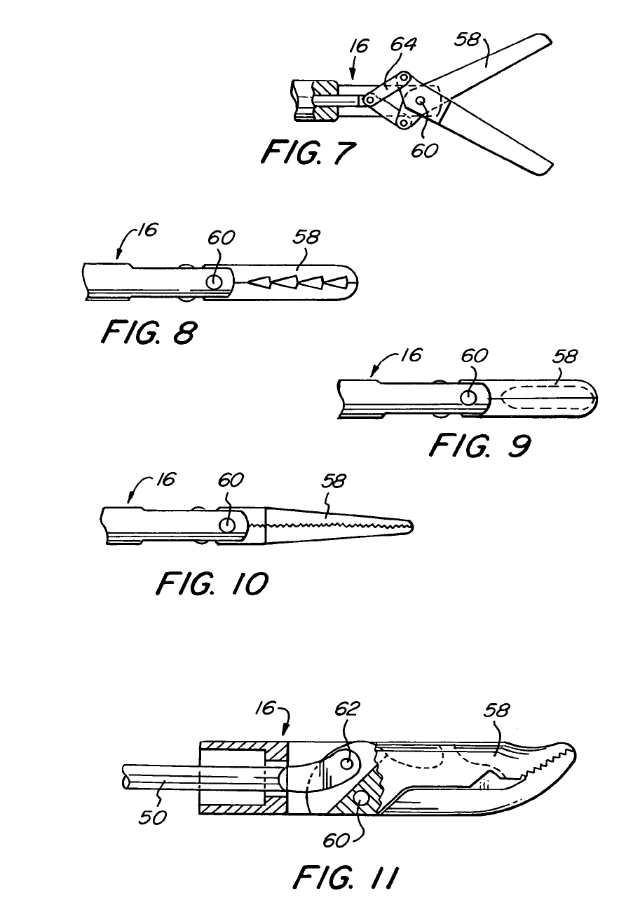

Referring now to FIGS. 4-11, various embodiments of working parts 16 are shown. In general, each working part 16 includes two jaw elements 58 which are pivotable with respect to each other about a pivot pin 60. Both jaw elements 58 may be moveable (i.e., with respect to insertion portion 14), or one jaw element 58 may be fixed while the other jaw element is moveable. Relative movement between the jaw elements 58 is caused by longitudinal reciprocal movement of the force transmission member 50 caused by actuation of handle portion 12 as described above. As is known in the art, various techniques may be employed for translating the longitudinal reciprocal movement of the force transmission member 50 to respective pivoting movement of the jaw elements 58 with respect to each other, such as by disposing pivot pin 60 as well as a pivoting connection 62 between one of jaw elements 58 and force transmission member 50 eccentrically (i.e., off of a longitudinal axis of force transmission member 50), such as shown in FIGS. 5B and 11, or by providing one (FIG. 6B) or more (FIG. 7) intermediate force transmission members 64.

Working part 16 may be attached to insertion portion 14 by way of a bayonet connection 66, by a threaded connection, or by any other appropriate means. Thus, working part 16 may be disassembled from insertion portion 14 for cleaning and disinfecting. In addition, the elements of working part 16 itself may be disassembled to further facilitate cleaning and disinfecting.

In general, the configuration of jaw elements 58 dictates the intended use of the working part 16, and thereby of the endoscopic surgical instrument 10 overall. For example, jaw elements 58 may be configured as graspers (FIGS. 4-6B), scissors (FIG. 7), gripper tools (FIG. 8), biopsy tools (FIG. 9), dissectors (FIG. 10), needle holders (FIG. 11), or any of other of various tools having a similar operation. The jaw elements 58, as well as various other portions of working part 16 may be made of any of numerous materials, such as metal, plastic, ceramic, etc.

Each of the working parts 16 falls into one of at least two subsets of working parts 16, with each subset of working parts 16 being visually distinctive by color with respect to the other subset of working parts 16. The subsets of working parts 16 may be based, for example, on the intended use of the working part 16 of each endoscopic surgical instrument 10. For example, working parts 16 which are configured as graspers (FIGS. 4 - 6B) may be colored black, working parts 16 which are configured as scissors (FIG. 7) may be colored gold, working parts 16 which are configured as gripper tools (FIG. 8) may be colored silver, working parts 16 which are configured as biopsy tools (FIG. 9) may be colored blue, working parts 16 which are configured as dissectors (FIG. 10) may be colored red, working parts 16 which are configured as needle holders (FIG. 11) may be colored green, and various other types of working parts 16 may have various other colors.

As another example, working parts which are intended for use in different surgeries and/or which are intended for use on different types of patients may be colored different colors. For example, instruments which form part of an arthroscopy “kit” may be colored one color, while instruments which form a part of a cardiac surgery “kit” may be colored another color. Similarly, instruments which are intended for use with children may be colored one color, while instruments which are intended for use with adults may be colored another color.

The entire working part 16 may be colored in the visually distinctive color, although it may be sufficient for only portions thereof to be so colored. For example, it may be only jaw elements 58 which are color coded. The visually distinctive coloring may be provided in any of numerous ways. For example, the color may be formed into the material (e.g., metal, plastic, ceramic) from which the working part 16 itself is made, or the color may be applied, such as using a paint or other coating. Different shades of the same color may be used (e.g., light blue and dark blue), so long as one is readily able to distinguish between the two shades.

The present invention, therefore, provides an endoscopic surgical instrument which is readily distinguishable as a particular desired endoscopic surgical instrument or type of endoscopic surgical instrument from among other endoscopic surgical instruments provided together on a table or tray or the like, which is readily identifiable as a particular endoscopic surgical instrument or type of endoscopic surgical instrument while it is inserted into a patient's body, and which includes parts which are readily distinguishable from the parts of other endoscopic surgical instruments during reassembly, after the endoscopic surgical instrument, along with other endoscopic surgical instruments, have been disassembled and cleaned and disinfected.

Although the invention has been described with reference to a particular arrangement of parts, features and the like, these are not intended to exhaust all possible arrangements or features, and indeed many other modifications and variations will be ascertainable to those of skill in the art.

What is claimed is:

1. An endoscopic surgical instruments system comprising a plurality of instruments, each instrument comprising:
an insertion portion adapted to be inserted into a body, said insertion portion having a proximal end extending outside the body during use and a distal end located within the body during use;
a working part attached to the distal end of said insertion portion, said working part being disposed within the body during use;
a handle portion attached to the proximal end of said insertion portion, said handle portion being disposed outside of the body during use, said handle portion being actuatable to actuate said working part via a force transmission member, and
wherein said system comprises a first kit comprising a first subset of said plurality of instruments, said first kit comprising a plurality of instruments required to perform a first type of surgery;
wherein all of the instruments comprising said first kit have working parts visually indistinguishable from each other by color;
wherein said system comprises a second kit comprising a second subset of said plurality of instruments, said second kit comprising a plurality of instruments required to perform a second type of surgery different than said first type of surgery;
wherein all of the instruments comprising said second kit have working parts visually indistinguishable from each other by color;
wherein all of the instruments comprising said first kit have working parts visually distinguishable from the working parts of all of the instruments comprising said second kit by color; and
wherein said plurality of instruments comprising said first kit are different than said plurality of instruments comprising said second kit.

2. The plurality of instruments of claim 1 wherein each said working part is formed from a material, and wherein at least a portion of each said working part of each subset of said plurality of instruments is visually indistinguishable from at least a portion of each other said working part of the same subset of said plurality of instruments by a color of the material.

3. The plurality of instruments of claim 2 wherein the material comprises at least one of a metal, a plastic and a ceramic.

4. The plurality of instruments of claim 1 wherein at least a portion of each said working part of each subset of said plurality of instruments is visually indistinguishable from at least a portion of each other said working part of the same subset of said plurality of instruments by a color which said working part is painted.

5. The plurality of instruments of claim 1 wherein an entirety of each said working part of each subset of said plurality of instruments is visually indistinguishable from an entirety of each other said working part of the same subset of said plurality of instruments by color.

6. The plurality of instruments of claim 1 wherein less than an entirety of each said working part of each subset of said plurality of instruments is visually indistinguishable from less than an entirety of each other said working part of the same subset of said plurality of instruments by color.

7. The plurality of instruments of claim 1 wherein all of the instruments of said first kit comprises a first type of instrument selected from the group consisting of: graspers, scissors, gripper tools, biopsy tools, dissectors, and needle holders; and
wherein all of the instruments of said second kit comprises a second type of instrument different than said first type of instrument selected from the group consisting of: graspers, scissors, gripper tools, biopsy tools, dissectors, and needle holders.

8. An endoscopic surgical instrument system comprising a plurality of instruments, each instrument comprising:
an insertion portion adapted to be inserted into a body, said insertion portion having a proximal end extending outside the body during use and a distal end located within the body during use;
a working part attached to the distal end of said insertion portion, said working part being disposed within the body during use;
a handle portion attached to the proximal end of said insertion portion, said handle portion being disposed outside of the body during use, said handle portion being actuatable to actuate said working part via a force transmission member, and
wherein said system comprises a first kit comprising a first subset of said plurality of instruments, said first kit comprising a plurality of instruments required for a first patient type;
wherein all of the instruments comprising said first kit have working parts visually indistinguishable from each other by color;
wherein said system comprises a second kit comprising a second subset of said plurality of instruments, said second kit comprising a plurality of instruments required for a second patient type different than said first patient type;
wherein all of the instruments comprising said second kit have working parts visually indistinguishable from each other by color;
wherein said plurality of instruments comprising the first kit are different than said plurality of instruments comprising said second kit;
wherein all of the instruments comprising said first kit have working parts visually distinguishable from the working parts of all of the instruments comprising said second kit by color.

9. The plurality of endoscopic surgical instruments of claim 8 wherein each said working part is formed from a material, and wherein at least a portion of each said working part of each subset of instruments is visually indistinguishable from at least a portion of each other said working part of the same subset instruments by a color of the material.

10. The plurality of instruments of claim 9 wherein the material comprises at least one of a metal, a plastic and a ceramic.

11. The plurality of instruments of claim 8 wherein at least a portion of each said working part of each subset of said plurality of instruments is visually indistinguishable from at least a portion of each other said working part of the same subset of said plurality of instruments by a color which said working part is painted.

12. The plurality of instruments of claim 8 wherein an entirety of each said working part of each subset of said plurality of instruments is visually indistinguishable from an entirety of each other said working part of the same subset of said plurality of instruments by color.

13. The plurality of instruments of claim 8 wherein less than an entirety of each said working part of each subset of said plurality of instruments is visually indistinguishable from less than an entirety of each other said working part of the same subset of said plurality of instruments by color.

14. The plurality of instruments of claim 8 wherein all of the instruments of said first kit comprise a first type of instruments selected from the group consisting of: graspers, scissors, gripper tools, biopsy tools, dissectors, and needle holders; and
wherein all of the instruments of said second kit comprise a second type of instruments different than said first type of instrument selected from the group consisting of: graspers, scissors, gripper tools, biopsy tools, dissectors, and needle holders.

15. An endoscopic surgical instrument system comprising a plurality of instruments, each instrument comprising:
an insertion portion adapted to be inserted into a body, said insertion portion having a proximal end extending outside the body during use and a distal end located within the body during use;
a working part attached to the distal end of said insertion portion, said working part being disposed within the body during use;
a handle portion attached to the proximal end of said insertion portion, said handle portion being disposed outside of the body during use, said handle portion being actuatable to actuate said working part via a force transmission member, and
wherein said system comprises a first kit comprising a first subset of said plurality of instruments, said first kit comprises a first type of instrument selected from the group consisting of: graspers, scissors, gripper tools, biopsy tools, dissectors, and needle holders;
wherein all of the instruments comprising said first kit have working parts visually indistinguishable from each other by color; and
wherein said system comprises a second kit comprising a second subset of said plurality of instruments, said second kit comprises a second type of instrument different than said first type of instrument selected from the group consisting of: graspers, scissors, gripper tools, biopsy tools, dissectors, and needle holders;
wherein all of the instruments comprising said second kit have working parts visually indistinguishable from each other by color;
wherein said plurality of instruments comprising the first kit have working parts visually distinguishable from the working parts of all the instruments comprising said second kit by color.
